(19) United States
(12) Patent Application Publication
(10) Pub. No.: US 2013/0118234 A1
(43) Pub. Date: May 16, 2013

(54) APPARATUS FOR MEASURING AIR AMOUNT OF ENGINE OIL AND METHOD USING THE SAME

(71) Applicant: Byung Sun Jeon, Whasung-Si (KR)

(72) Inventor: Byung Sun Jeon, Whasung-Si (KR)

(73) Assignees: Kia Motors Corp., Seoul (KR); Hyundai Motor Company, Seoul (KR)

(21) Appl. No.: 13/494,513

(22) Filed: Jun. 12, 2012

(30) Foreign Application Priority Data
Nov. 16, 2011 (KR) 10-2011-0119754

(51) Int. Cl. G01N 33/30

(52) U.S. Cl. 73/53.05

(57) ABSTRACT

An apparatus for measuring air amount of engine oil includes an inflow pipe in which the engine oil flows in connection with an engine and an inflow valve, a measuring burette having an inflow hole connected with the inflow pipe and an outflow hole formed at the other side and a measurement camera provided at the side of the measuring burette to photograph graduations, a vent pipe branched from the outflow hole of the measuring burette and a bent valve provided on the vent pipe, a drain burette having a drain pipe connected with the outflow hole provided at one side thereof and having a drain valve provided on the drain pipe, an auxiliary drain pipe having one side branched from the inflow hole and the other side connected to the drain burette and an auxiliary valve provided on the auxiliary drain pipe, and a control unit.

### CROSS-REFERENCE TO RELATED APPLICATION

The present application claims priority of Korean Patent Application Number 10-2011-0119754 filed Nov. 16, 2011, the entire contents of which application is incorporated herein for all purposes by this reference.

### BACKGROUND OF INVENTION

1. Field of Invention

The present invention relates to an apparatus for measuring an air amount of engine oil which in which test efficiency and reliability for a test result are improved, there are no problems associated with high expense and noise caused by the use of complicated equipment, and can be used without a limit in oil temperature, and a method using the same.

2. Description of Related Art

During a circulation process of an oil in an engine, the oil has various pressure conditions from atmospheric pressure up to high-pressure states of a level of 4 to 5 bars. Further, before an oil that completes a role in each part in the engine flows into an oil pump, the oil contacts air. In an appropriately designed lubrication system, an air that takes in through the process is not generally problematic, but when any problem occurs in design or when a design used section is deviated, the air excessively flows in the oil, and as a result, a problem may occur.

Meanwhile, when the air excessively flows in the oil, problems may occur in various parts. In the case of a bearing, the oil should support a load in addition to lubrication under a high-pressure condition, but a load supporting capability may be deteriorated due to the air in the oil to cause the bearing to be damaged.

Further, desired force cannot be acquired in a mechanism using oil pressure such as a CVVT. Under a relatively low-pressure condition, the oil gets out of from the oil as bubbles to cause a sponge phenomenon in which a valve cannot be normally pressed even though a cam is pressed due to air generated from a mechanism such as an HLA and generates large noise.

In order to prevent the problem, a process configured to select a minimum oil capacity and a maximum oil capacity by measuring an air amount while the engine is developed and to have an appropriate level within the selected oil capacities is required.

In the case of measuring the air amount, an operator directly takes out oil in a high-temperature state in the engine that rotates at high speed into a container to separate the oil and the air from each other, thereby measures the air amount or an oil amount. However, an operation under high speed and high temperature is very dangerous and in particular, a development engine which may be broken within several times has a serious safety-related problem.

In order to evade the serious problem, various measurement methods are provided and commercial products using the measurements are also launched. However, as shown in the next chapter, since each method has its own problem, each method is still dangerous, but a basic measurement method is used.

As one of various measurement methods in the related art, a method of pressurizing and measuring collected oil with 1 bar by using a piston again after separating oil and air in a vacuum state by using the collected oil, but vacuum forming equipment and pressurizing equipment are required and air separation efficiency depends on a vacuum level and precision of the pressurizing equipment and a maximum measurement temperature is limited to 120° C. or so.

Further, a method of analyzing a compression curve by pressurizing oil at 200 bar after collecting oil in a compression chamber is provided, but a compressor for pressurizing oil up to 200 bar is required and noise in compression is generated and a change of a characteristic (compression rate) depending on an oil deterioration level is not considered and can be measured only up to 15%.

Further, a method of calculating the air amount by measuring the density by transmitting X-rays to oil and bubbles in the oil are provided, but appropriate shielding of the X-rays is required, a dissolved air amount in the oil cannot be calibrated, oil mixed with air of 0% as a reference value is required, and a measurement value swings depending on the size of the passing bubble.

Meanwhile, a method of calculating the air amount by forming an additional path and measuring oil passing through the path by using an hydrometer, a thermometer, and a manometer is provided, but changes of characteristics (density and compression rate) depending on the oil deterioration level is not considered, the dissolved air amount in the oil is not calibrated, and the oil mixed with air of 0% as the reference value is required.

The information disclosed in this Background section is only for enhancement of understanding of the general background of the invention and should not be taken as an acknowledgement or any form of suggestion that this information forms the prior art already known to a person skilled in the art.

### SUMMARY OF INVENTION

Various aspects of the present invention provide for an apparatus for measuring an air amount of engine oil which in which test efficiency and reliability for a test result are improved, there are no problems associated with high expense and noise caused by the use of complicated equipment, and can be used without any limit in oil temperature, and a method using the same.

Various aspects of the present invention provide for an apparatus for measuring an air amount of engine oil, including an inflow pipe in which the engine oil flows in connection with an engine and an inflow valve provided therein, a measuring burette having an inflow hole connected with the inflow pipe formed at one side and an outflow hole 340 formed at the other side and a measurement camera provided at the side of the measuring burette to photograph graduations, a vent pipe branched from the outflow hole of the measuring burette and a bent valve provided on the vent pipe, a drain burette having a drain pipe connected with the outflow hole provided at one side thereof and having a drain valve provided on the drain pipe, an auxiliary drain pipe having one side branched from the inflow hole and the other side connected to the drain burette and an auxiliary valve provided on the auxiliary drain pipe, and a control unit controlling the valves.

The control unit opens the inflow valve and the drain valve in inflow control of the engine oil, opens the vent valve after closing the inflow valve and the drain valve in measurement control, and opens the vent valve and the auxiliary valve in ending control to control the engine oil to be discharged to the drain burette.

An outflow pipe connected with the engine may be provided in the outflow hole of the measuring burette, a first outflow valve may be provided on the outflow pipe, the drain pipe may be branched from a rear end of the first outflow valve of the outflow pipe, and a second outflow valve may be provided between the engine and a branch point of the drain pipe.

The control unit opens the inflow valve and the first outflow valve and opens the drain valve or the second outflow valve in the inflow control of the engine oil, opens the vent valve after closing the inflow valve and the first outflow valve in the measurement control, and opens the vent valve and the auxiliary valve in the ending control to control the engine oil to be discharged to the drain burette.

The apparatus may further include an auxiliary camera provided on the side of the drain burette to photograph the graduations.

Meanwhile, other aspects of the present invention provide for a method for measuring an air amount of engine oil using the measurement apparatus, including an inflow process of allowing the engine oil to flow in the measuring burette by opening the inflow valve and the drain valve, a preparation process of storing the engine oil in the measuring burette by closing the inflow valve and the drain valve, a measurement process of discharging air in the engine oil stored in the burette by opening the vent valve and photographing graduations before and after discharging the air, and an ending process of discharging the engine oil to the drain burette by opening the auxiliary valve.

Further, other aspects of the present invention provide for a method for measuring an air amount of engine oil using the measurement apparatus, including a selection process of opening the drain valve when the engine oil after the measurement is drained and previously opening the second outflow valve when the engine oil is sent to the engine again, an inflow process of allowing the engine oil to flow in the measuring burette by opening the inflow valve and the first outflow valve, a preparation process of storing the engine oil in the measuring burette by closing the inflow valve and the first outflow valve, a measurement process of discharging air in the engine oil stored in the measuring burette by opening the vent valve and photographing graduations before and after discharging air by using the measurement camera, and an ending process of discharging the engine oil to the outside of the drain burette by opening the auxiliary valve when the measuring burette is drained and closing the vent valve when the measuring burette is not drained.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is a diagram showing an exemplary apparatus for measuring an air amount of engine oil according to the present invention.

FIG. 2 is a flowchart of a measurement method using the apparatus for measuring an air amount of engine oil shown in FIG. 1.

It should be understood that the appended drawings are not necessarily to scale, presenting a somewhat simplified representation of various features illustrative of the basic principles of the invention.

In the figures, reference numbers refer to the same or equivalent parts of the present invention throughout the several figures of the drawing.

### DETAILED DESCRIPTION

Reference will now be made in detail to various embodiments of the present invention(s), examples of which are illustrated in the accompanying drawings and described below. While the invention(s) will be described in conjunction with exemplary embodiments, it will be understood that present description is not intended to limit the invention(s) to those exemplary embodiments. On the contrary, the invention(s) is/are intended to cover not only the exemplary embodiments, but also various alternatives, modifications, equivalents and other embodiments, which may be included within the spirit and scope of the invention as defined by the appended claims.

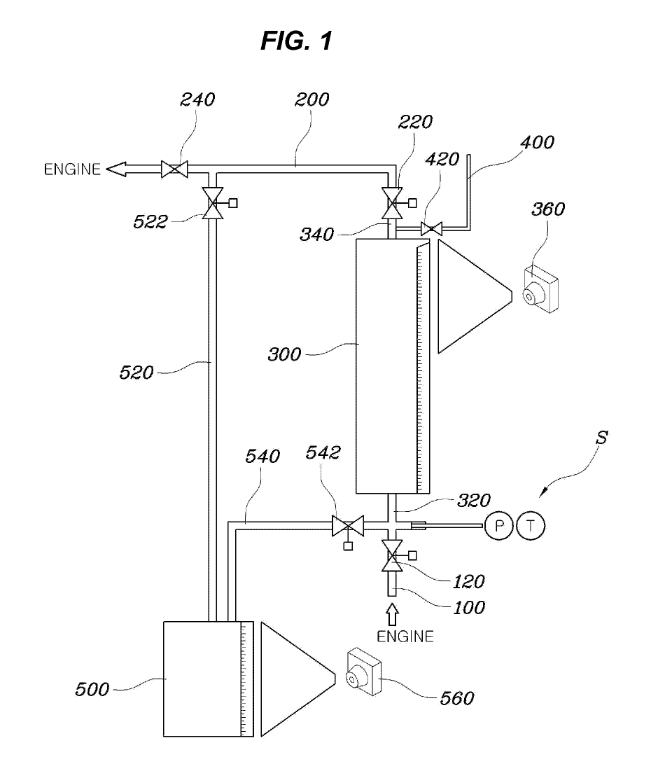

FIG. 1 is a diagram showing an apparatus for measuring an air amount of engine oil according to various embodiments of the present invention. The apparatus for measuring an air amount of engine oil according to various embodiments of the present invention includes an inflow pipe 100 in which the engine oil flows in connection with an engine and an inflow valve 120 provided therein, a measuring burette 300 having an inflow hole 320 connected with the inflow pipe 100 formed at one side and an outflow hole 340 formed at the other side and a measurement camera 360 provided at the side of the measuring burette 300 to photograph graduations, a vent pipe 400 branched from the outflow hole 340 of the measuring burette 300 and a bent valve 420 provided on the vent pipe 400, a drain burette 500 having a drain pipe 520 connected with the outflow hole 340 provided at one side thereof and having a drain valve 522 provided on the drain pipe 520, an auxiliary drain pipe 540 having one side branched from the inflow hole 320 and the other side connected to the drain burette 500 and an auxiliary valve 542 provided on the auxiliary drain pipe 540, and a control unit controlling the valves.

Herein, the control unit opens the inflow valve 120 and the drain valve 522 in inflow control of the engine oil, opens the vent valve 420 after closing the inflow valve 120 and the drain valve 522 in measurement control, and opens the vent valve 420 and the auxiliary valve 542 in ending control to control the engine oil to be discharged to the drain burette 500.

That is, the apparatus for measuring an air amount of engine oil according to various embodiments of the present invention is provided with a plurality of pipes and valves. The valves, which are initially closed, may be opened only by control of the control unit. Therefore, individual components and engines may be protected even by a misoperation of a hydraulic system.

Meanwhile, the measurement camera is installed in the measuring burette to accurately check the amount of air by viewing the camera, and as a result, an operator can be protected through measurement and observation in a remote place. Further, the camera can measure the amount of air more accurately when the control unit converts the graduations into data by analyzing images thereof.

Meanwhile, an outflow pipe 200 connected with the engine may be configured in the outflow hole 340 of the measuring burette 300, a first outflow valve 220 may be provided on the outflow pipe 200, the drain pipe 520 may be branched from a rear end of the first outflow valve 220 of the outflow pipe 200, and a second outflow valve 240 may be provided between the engine and a branch point of the drain pipe 520.

By this configuration, the control unit finally opens the inflow valve 120 and the first outflow valve 220 and opens the drain valve 522 or the second outflow valve 240 in the inflow control of the engine oil, opens the vent valve 420 after closing the inflow valve 120 and the first outflow valve 220 in the measurement control, and opens the vent valve 420 and the auxiliary valve 542 in the ending control to control the engine oil to be discharged to the drain burette 500.

In addition, the apparatus for measuring an air amount of engine oil further includes an auxiliary camera 560 provided on the side of the drain burette 500 to photograph the graduations to accurately determine the amount of drained oil in the remote place, thereby preventing the oil from leaking.

A method for measuring an air amount of engine oil, which uses the measurement apparatus of various embodiments of the present invention includes, an inflow process of allowing the engine oil to flow in the measuring burette by opening the inflow valve and the drain valve, a preparation process of storing the engine oil in the measuring burette by closing the inflow valve and the drain valve, a measurement process of discharging air in the engine oil stored in the burette by opening the vent valve and photographing graduations before and after discharging the air, and an ending process of discharging the engine oil to the drain burette by opening the auxiliary valve.

More specifically, the method includes a selection process (S100) of opening the drain valve when the engine oil after the measurement is drained and previously opening the second outflow valve when the engine oil is sent to the engine again, an inflow process (S200) of allowing the engine oil to flow in the measuring burette by opening the inflow valve and the first outflow valve, a preparation process (S260) of storing the engine oil in the measuring burette by closing the inflow valve and the first outflow valve, a measurement process (S300) of discharging air in the engine oil stored in the measuring burette by opening the vent valve and photographing graduations before and after discharging air by using the measurement camera, and an ending process (S420 and S480) of discharging the engine oil to the outside of the drain burette by opening the auxiliary valve when the measuring burette is drained and closing the vent valve when the measuring burette is not drained.

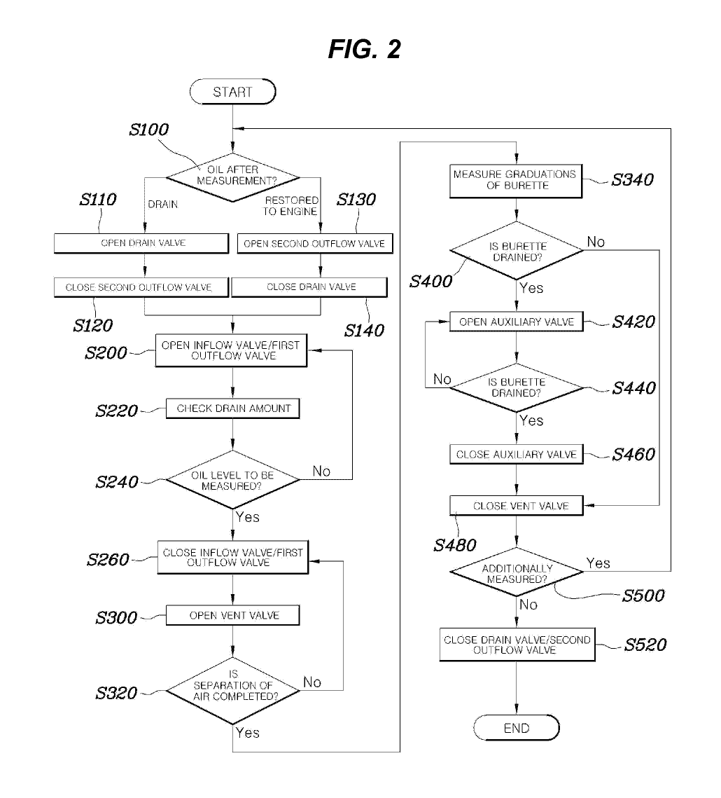

FIG. 2 is a flowchart of a measurement method using the apparatus for measuring an air amount of engine oil shown in FIG. 1. First, how to process oil after measurement is determined (S100). The oil may be discharged to the drain burette and restored to the engine again. As a result, when the oil is drained, the drain valve is opened (S110) and when the oil is sent to the engine again, the second outflow valve is previously opened (S130) and the rest of the valves are closed (S120 and S140).

Thereafter, the engine oil flows in the measuring burette by opening the inflow valve and the first outflow valve (S200). When a measurement level is satisfied while viewing the graduations of the measuring burette through the measurement camera, the engine oil is stored in the measuring burette by closing the inflow valve and the first outflow valve (S220, S240, and S260).

Thereafter, the air in the engine oil stored in the measuring burette is discharged by opening the bent valve and the graduations before and after discharging the air are photographed by using the measurement camera (S300, S320, and S340). Therefore, engine oil of a desired amount is collected to accurately measure the amount of air included in the engine oil.

Thereafter, when the measuring burette intends to be drained (S400), the engine oil is discharged to the drain burette by opening the auxiliary valve (S420 and S440). In addition, when the discharging is completed, the auxiliary valve and the vent valve are closed (S460 and S480).

Further, when the measuring burette does not intend to be drained, only the vent valve is closed (S480). Then, when next oil intends to be continuously measured, the above process is performed from the first again (S500). The drain valve and the second outflow valve are closed when the measurement ends (S520).

Through the above process, air is discharged by collecting the oil in the engine only with a desired amount and the corresponding amount is measured and the oil may be collected in the drain and restored to the engine again. Further, the graduations are measured in the drain in a burette manner and the graduations are also photographed with the camera to be provided, and as a result, no oil leaks or flows out during the measurement process. Therefore, it is easy to manage the overall amount of oil which is measured and it is easy to determine the amount of air compared with the total amount.

Moreover, in this method, since the overall oil can be measured dividually according to a temporal sequence, it is very useful to check a change of the amount of air to the overall oil and remaining oil is also thoroughly managed to accurately determine the amount of air compared with the total amount.

According to an apparatus for measuring an air amount of engine oil, which has the above structure and a method using the same, test efficiency and reliability for a test result are improved, there is no problem associated with high expense and noise caused by the use of complicated equipment, and can be used without a limit in oil temperature, and a method using the same.

Further, oil mixed with air of 0% as a reference value is not required, a conversion through a complicated equation is not required, and oil does not leak, and an accurate air amount and the amount of remaining oil other than the air can be accurately measured.

For convenience in explanation and accurate definition in the appended claims, the terms upper or lower, front or rear, inside or outside, and etc. are used to describe features of the exemplary embodiments with reference to the positions of such features as displayed in the figures.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. An apparatus for measuring an air amount of engine oil of an engine, comprising:
an inflow pipe in which the engine oil flows having an inflow valve provided therein;
a measuring burette having an inflow hole connected with the inflow pipe formed at one side and an outflow hole formed at another side;
a measurement camera provided at the measuring burette to photograph graduations;
a vent pipe branched from the outflow hole of the measuring burette and a bent valve provided on the vent pipe;
a drain burette having a drain pipe connected with the outflow hole provided at one side thereof and having a drain valve provided on the drain pipe;
an auxiliary drain pipe having one side branched from the inflow hole and another side connected to the drain burette and an auxiliary valve provided on the auxiliary drain pipe; and
a control unit controlling the valves.

2. The apparatus for measuring an air amount of engine oil of claim 1, wherein the control unit opens the inflow valve and the drain valve in inflow control of the engine oil, opens the vent valve after closing the inflow valve and the drain valve in measurement control, and opens the vent valve and the auxiliary valve in ending control to control the engine oil to be discharged to the drain burette.

3. The apparatus for measuring an air amount of engine oil of claim 1, wherein an outflow pipe connected with the engine is provided in the outflow hole of the measuring burette, a first outflow valve is provided on the outflow pipe, the drain pipe is branched from a rear end of the first outflow valve of the outflow pipe, and a second outflow valve is provided between the engine and a branch point of the drain pipe.

4. The apparatus for measuring an air amount of engine oil of claim 3, wherein the control unit opens the inflow valve and the first outflow valve and opens the drain valve or the second outflow valve in the inflow control of the engine oil, opens the vent valve after closing the inflow valve and the first outflow valve in the measurement control, and opens the vent valve and the auxiliary valve in the ending control to control the engine oil to be discharged to the drain burette.

5. The apparatus for measuring an air amount of engine oil of claim 1, further comprising an auxiliary camera provided on the side of the drain burette to photograph the graduations.

6. A method for measuring an air amount of engine oil using the measurement apparatus of claim 1, comprising:
an inflow process of allowing the engine oil to flow in the measuring burette by opening the inflow valve and the drain valve;
a preparation process of storing the engine oil in the measuring burette by closing the inflow valve and the drain valve;
a measurement process of discharging air in the engine oil stored in the burette by opening the vent valve and photographing graduations before and after discharging the air; and
an ending process of discharging the engine oil to the drain burette by opening the auxiliary valve.

7. A method for measuring an air amount of engine oil using the measurement apparatus of claim 3, comprising:
a selection process of opening the drain valve when the engine oil after the measurement is drained and previously opening the second outflow valve when the engine oil is sent to the engine again;
an inflow process of allowing the engine oil to flow in the measuring burette by opening the inflow valve and the first outflow valve;
a preparation process of storing the engine oil in the measuring burette by closing the inflow valve and the first outflow valve;
a measurement process of discharging air in the engine oil stored in the measuring burette by opening the vent valve and photographing graduations before and after discharging air by using the measurement camera; and
an ending process of discharging the engine oil to the outside of the drain burette by opening the auxiliary valve when the measuring burette is drained and closing the vent valve when the measuring burette is not drained.
